(19) United States
(12) Patent Application Publication
(10) Pub. No.: US 2005/0288616 A1
(43) Pub. Date: Dec. 29, 2005

(54) Sampling swab

(75) Inventors: Richard H. Bozenbury JR., North Plainfield, NJ (US); Reno F. Debono, Clinton, NJ (US); Ludmila L. Danylewych-May, North York (CA); Lucy Fricano, Ancaster (CA); Lena Kim, Richmond Hill (CA)

Correspondence Address: FOLEY AND LARDNER LLP;SUITE 500, 3000 K STREET NW, WASHINGTON, DC, 20007, US

(73) Assignee: SMITHS DETECTION, INC.

(21) Appl. No.: 10/876,478

(22) Filed: Jun. 28, 2004

(51) Int. Cl. A61M 35/00

(52) U.S. Cl. 604/1

(57) ABSTRACT

A sampling swab useful in trace analyte detection is provided. The sampling swab possesses absorption/adsorption and desorption properties suitable for use trace analyte sample collection. The sampling swab is also capable of withstanding repeated mechanical stress and heat treatment. Methods for producing a sampling swab that is substantially free of impurities that interfere with analyte detection, but which remains sufficiently resistant to degradation by mechanical and heat stress are also provided.

### BACKGROUND

The present invention relates to a sampling swab and methods of making a sampling swab. Specifically, the invention relates to sampling swabs and methods of making sampling swabs useful in trace analyte detection techniques.

Trace analyte detection is the detection of small amounts of analytes, often at nanogram to picogram levels. Trace analyte detection has a numerous applications, such as screening individuals and baggage at transportation centers, mail screening, facility security applications, military applications, forensics applications, narcotics detection and identification, cleaning validation, quality control, and raw material identification. Trace analyte detection can be particularly useful for security applications such as screening individuals or items for components in explosive materials, narcotics or biological contaminants where small amounts of these components are deposited on the individual or on the outside of a package or bag.

A variety of different techniques can be used for trace analyte detection. These methods include ion mobility spectrometry (IMS), mass spectrometry, gas chromatography, liquid chromatography, and high performance liquid chromatography (HPLC).

IMS is a particularly useful technique for rapid and accurate detection and identification of trace analytes such as narcotics, explosives, and chemical warfare agents. The fundamental design and operation of an ion mobility spetectometer is addressed in, for example, Ion Mobility Spectrometry (G. Eiceman and Z. Karpas, CRC Press, Boca Raton, Fla., 1994). IMS detects and identifies known analytes by detecting a signal which is unique for each analyte. IMS measures the drift time of ions through clean, dry ambient air at atmospheric pressure. Analysis of analytes in a sample begins with collection of a sample and introduction of the sample into the spectrometer. A sample is heated to transform analyte from solid, liquid or vapor preconcentrated on a particle into a gaseous state. Analyte molecules are ionized in the reaction region of the IMS detector. Ions are then spatially separated in the IMS drift region in accordance to their ion mobility, which is an intrinsic property of an ion. In an IMS detector, where only singly charged ions are typically formed, ion mobility is roughly directly proportional to ion mass. An induced current at the collector generates a signature for each ion as a function of the time required for that ion to reach the collector. This signature is used to identify a specific analyte.

A variety of different methods can be used to introduce a sample into a detection instrument and the method will depend, in part, on the type of sample being analyzed and the detection technique. For example, U.S. Pat. Nos. 6,442,997, 6,073,499, 5,859,362, and 5,162,652 disclose devices for collecting vapor or air samples, U.S. Pat. No. 6,073,498 discloses a device for collecting fluid samples, U.S. Pat. No. 5,037,611 is directed to a method adsorbing gaseous samples on a tape, and U.S. Pat. No. 5,741,984 discloses a method which introduces a sample from a finger by pressing the finger on a sampling “token.” U.S. Pat. Nos. 5,859,375 and 5,988,002 are directed to a methods and apparatus for collecting samples using a hand-held sampling device.

Another sampling method involves contacting an object or other substrate to be tested with a textile sampling swab which collect analyte particles. Upon contact of a sampling swab with a substrate to be tested, solid sample particles can become imbedded into the porous structure of the textile swab. If the sample is in liquid form, the liquid can absorb into the fibers of the swab. In IMS, the swab is placed into the detection instrument and the sample thermally desorbed from the swab. A swab for use in IMS should have absorption and desorption properties suitable for the analytes and substrates to be sampled, should be compatible with the geometry and processes performed by the instrument, should be durable and stable over a range of temperatures, and should be substantially free from contaminants and impurities capable in interfering with sample analysis.

A sampling swab should have the ability to absorb and/or adsorb an analyte upon contact with the swab, as well as efficiently desorb the analyte from the swab upon placement of the swab in a detection instrument. For example, a sampling swab should be able to effectively absorb/adsorb volatile substances into its fibrous structure or embed sample particles into its porous structure upon contact with an analyte present on the test surface. Additionally, a sampling swab should not interfere with a desorption process of a sample analyte from its surface or fibers during desorption of the collected sample.

A suitable swab also should be durable and stable, capable of resisting decomposition and degradation due to heating and mechanical stress. Decomposition and degradation of a swab can lead to contamination of the detection instrument, thus compromising the integrity of the analysis and potentially fouling the detection instrument. Decomposed and degraded fibers can generate false positives or can interfere with analyte detection resulting in failure in detecting an analyte. In addition, decomposed and degraded fibers can remain in the detection instrument, thus compromising subsequent analyses and risking damage to the detection instrument. The resistance of a swab to decomposition and degradation is affected by physical properties of materials used, such as fiber strength, fiber length, fiber diameter, and smoothness of sampling swab fabric.

The stability of a textile fiber at high temperatures is particularly important in detection methods involving heating the swab. For example, in ion mobility spectrometry, the swab is heated to desorb and vaporize analyte molecules collected by contact of the swab with a substrate being tested. Thus, it is desirable for the swab to resist decomposition and degradation at temperatures in excess of 300° C. for durations of at least one minute.

It is also desirable that a swab is substantially free of impurities which may interfere with the detection of analytes. Unprocessed cellulosic sampling swab material will contain substances found in natural fibers, such as waxes, natural oils and starches as well as impurities introduced during the manufacturing process, such as sizing agents and lubricants These impurities can interfere with the analyte detection by creating unacceptable background signal which swamps out analyte signal and can also cause instrument contamination and instrument failure.

Other potential sources of contamination to both sample and detection instrument are “trash” and “neps.” “Trash” or “extractables” refers to non-lint materials trapped in the cotton, such as leaf, bark, seedcoat fragments, dust, and oil. The amount of trash or extractables is affected by plant variety, harvesting methods, and harvesting conditions. The amount of trash or extractables remaining after ginning depends on the amount present prior to ginning, and on the type and amount of cleaning and drying equipment used. However, even with the most careful harvesting and ginning methods, a small amount of trash can remain in the fiber lint. Trash or extractables can be released from the swab resulting in both compromised sample analysis and fouling of the instrument itself.

A nep is a small tangled fiber knot often caused by processing of cellulosic textiles. Harsh mechanical or chemical processes can result in nep formation in the a fiber or fabric due to damage and weakening of the cellulose. A nep which extends beyond the horizontal plant of a swab can become dislodged or weakened when stressed mechanically while contacting or rubbing a swab on a substrate to be tested.

A variety of methods are known for removing non-cellulosic components found in native cellulosic fiber and fiber textiles after processing and for altering textile properties after processing. These processes, referred to as “scouring” remove fats, waxes, starches and other non-cellulosic materials from the native fiber and processed textile. During scouring, natural waxes and fats in and on the cellulosic fibers are saponified and pectins and other non-cellulosic materials are released, such that impurities can be removed by rinsing the fiber with water.

A variety of different enzymatic and chemical scouring and preparation agents are known. For example, U.S. Pat. No. 4,076,500 discloses a process for scouring cotton involving contacting the cotton with an alkaline compound in a chlorinated solvent, U.S. Pat. No. 4,312,634 discloses scouring cellulosic materials in an alkaline bath at elevated temperatures, and U.S. Pat. No. 4,796,334 discloses a method of rendering these adhesive honeydew droplets non-adhesive by contacting the fibers with a heating plate at a predetermined temperature for a predetermined time.

Enzymes are used in the textile industry and various uses are disclosed in the literature. Enzymes commonly used include amylases, cellulases, pectinases and lipases. In typical applications, amylases are used to remove sizing agents, cellulases are used to alter the surface finish of, or remove impurities from, cotton fibers and lipases are used to remove fats and oils from the surface of natural fibers (e.g., cotton, silk, etc.). For example, U.S. Pat. Nos. 6,551,358 and 5,912,407 disclose methods of scouring cotton with the enzyme pectinase. U.S. Pat. No. 6,630,342 is directed to biopreparation of cellulosic fibers at high temperatures using themostable pectate lyases.

Chemical scouring agents, typically alkalis, are generally applied at high temperatures, often above the boiling point of water. Because this type of scouring is harsh on cellulosic fibers and can cause oxidation to form oxycellylose, the cotton fiber may be damaged during the scouring process. See, e.g., Grunig, Colourage, Jul. 19, 1982, pp. 3-11. Such harsh processes can have a detrimental influence on the strength of the cellulosic fiber. Similarly, enzymatic scouring and preparation methods can also result in weakened fibers in certain protocols.

The methods necessary to product a sampling swab that is substantially free of impurities that interfere with analyte detection are harsh and result in weakening of the underlying cellulose component of the fabric or fiber. The negative impact of harsh cleaning methods on the resulting fabric produce an inferior swab for the purposes of sample collection because the shortened and weakened fibers cannot withstand repeated use without degradation and deposition of dust, lint, free neps and other contaminating materials into a detection instrument. Thus, there is a need for a textile processing and cleaning protocol which results in a swab which is clean and while maintaining sufficient strength and structural integrity.

### SUMMARY OF THE INVENTION

Thus, there is need in the art for a sampling swab and a method of manufacturing a sampling swab, having absorption and analyte collection efficiency together with desorption properties suitable for trace analyte sample collection, which is capable of withstanding repeated mechanical stress and heat treatment.

In one embodiment, the invention provides a sampling swab comprising a cellulosic fabric comprising a thread count of at least 80×80, a weight per unit area of between approximately 0.01 to approximately 0.02 g/cm², a thickness of between approximately 0.01 to approximately 0.03 cm, and an air permeability of between approximately 80 to approximately 125 CFM. In one embodiment, a swab is 0.025 cm thick.

In another embodiment, the invention provides a method of processing cellulosic fabric comprising contacting a cellulosic fiber with a sizing agent, manipulating the cellulosic fiber into a cellulosic fabric, contacting the cellulosic fabric with a scouring agent, contacting the cellulosic fabric with a solvent and heating the cellulosic fabric to a temperature between 120° C. to 250° C. for a time of between 1 to 60 minutes.

In a further embodiment, the invention provides a sampling swab produced by steps comprising contacting a cotton fiber with a sizing agent, manipulating the cotton fiber into a cotton fabric, contacting the cotton fabric with a scouring agent, contacting the cotton fabric with a solvent, and heating the cotton fabric to a temperature between 120 to 250° C. for a time of between approximately 5 to 15 minutes.

These and other features, aspects, and advantages of the present invention will become apparent from the following description, appended claims, and the accompanying exemplary embodiments shown in the drawings, which are briefly described below.

It is to be understood that both the foregoing general description and the following detailed description are exemplary and explanatory only, and are not restrictive of the invention as claimed.

### BRIEF DESCRIPTION OF THE DRAWINGS

FIG. 1. Plasmagram of clean sampling swab obtained using IONSCAN® 400B IMS ion mobility spectrometer (Smiths Detection) run with following parameters: negative ionization mode, drift tube temperature of 111° C., inlet temperature of 240° C., desorber temperature of 225° C. The ionization reagent is hexachloroethane, the drift gas is cleaned, dried room air at a flow rate of 300 cm³/min. The scan period is 22 ms with a 0.200 ms shutter gate pulse, 0.025 s analysis delay, 6.600 s analysis duration, 20 co-added scans per segment, and 15 segments per analysis.

FIG. 2. Plasmagram of sampling swab doped with 600 pg TNT. Sample was run on and IONSCAN® 400B IMS ion mobility spectrometer (Smiths Detection) run with following parameters: negative ionization mode, drift tube temperature of 111° C., inlet temperature of 240° C., desorber temperature of 225° C. The ionization reagent is hexachloroethane, the drift gas is cleaned, dried room air at a flow rate of 300 cm³/min. The scan period is 22 ms with a 0.200 ms shutter gate pulse, 0.025 s analysis delay, 6.600 s analysis duration, 20 co-added scans per segment, and 15 segments per analysis.

FIG. 3. Plasmagram of sampling swab doped with 1 ng cocaine. Sample was run on an IONSCAN® 400B IMS ion mobility spectrometer (Smiths Detection) run with following parameters: positive ionization mode, drift tube temperature of 237° C., inlet temperature of 280° C., desorber temperature of 285° C. The ionization reagent is nicotinamide and drift gas is cleaned, dried room air at a flow rate of 300 cm³/min. The scan period is 20 ms with a 0.200 ms shutter gate pulse, 0.025 s analysis delay, 8.000 s analysis duration, 20 co-added scans per segment, and 20 segments per analysis.

### DETAILED DESCRIPTION

The invention provides a sampling swab with advantageous properties for sample collection in trace analyte detection. Qualities that impart the ability of a swab to function effectively include, but are not limited to sample collection efficiency, durability, and purity. These qualities are affected by physical properties of the sampling swab, including but not limited to porosity, density, thread count, fiber strength, fiber length, smoothness of swab surface, extractables content and stability at high temperatures.

Unless indicated otherwise, all technical and scientific terms are used in a manner that conforms to common technical usage. Generally, the nomenclature of this description and the described procedures and techniques are well known and commonly employed in the art. “Approximately,” as it is used herein, generally refers to a variation of 10% to 20% from a given value and is meant to allow for error inherent in measurement techniques as well as differences in measurement values that can be obtained when measurements are performed using different techniques.

A. Sampling Swab Uses and Performance Properties

A sampling swab of the present invention can be used for sample collection in any suitable trace detection technique. Suitable detection techniques include, but are not limited to IMS, mass spectrometry, and gas chromatography, liquid chromatography, and high performance liquid chromatography and combinations of these methods. In one embodiment, a swab is used to collect samples for IMS.

Sampling swabs of the present invention are useful collecting samples containing of a wide range of analytes, including but not limited to explosives, narcotics, chemical warfare agents, toxins and other chemical compounds. “Sample” refers, without limitation, to any molecule, compound or complex that is adsorbed, absorbed, or imbedded in the fibrous structure of a sampling swab. A sample can contain an analyte of interest, referred to herein as an “analyte” or “sample analyte,” which is understood to be any analyte the presence of which is to be detected using a detection technique.

Explosives which can be collected using a swab include, but are not limited to, 2-amino-4,6-dinitrotoluene, 4-amino-2,6-dinitrotoluene, ammonal, ammonium nitrate, black powder, 2,4-dimethyl-1,3-dinitrobutane, 2,4-dinitrotoluene, ethylene glycol dinitrate, forcite 40, GOMA-2, hexanitrostilbene, 1,3,5,7-tetranitro-1,3,5,7-tetrazacyclooctane (HMX), mononitrotoluene, nitroglycerine, pentaerythritol tetranitrate (PETN), 1,3,5-trinitro-1,3,5-triazacyclohexane (RDX), semtex-A, Semtex-H, smokeless powder, trinitro-2,4,6-phenylmethylnitramine tetryl (Tetryl), 2,4,6-trinitrotoluene (TNT), trilita, and 1,3,5-trinitrobenzene and combinations of these compounds. In one embodiment, the explosive which are collected are 1,3,5-trinitro-1,3,5-triazacyclohexane, pentaerythritol tetranitrate, 2,4,6-trinitrotoluene, trinitro-2,4,6-phenylmethylnitramine tetryl, nitroglycerine, ammonium nitrate, 3,5,7-tetranitro-1,3,5,7-tetrazacyclooctane, and combinations thereof.

Narcotics which can be collected using a swab include, but are not limited to 6-acetylmorphine, alprazolam, amobarbital, amphetamine, antipyrine, benzocaine, benzoylecgonine, bromazepam, butalbital, carbetapentane, cathinone, chloradiazepoxide, chlorpheniramine, cocaethylene, cocaine, codeine, diazepam, ecgonine, ecognine methyl ester (EME), ephedrine, fentanyl, flunitrazepam, hashish, heroin, hydrocodone, hydromorphone, ketamine, lidocaine, lorazepam, lysergic acid diethylamide (LSD), lysergic acid, N-methyl-1-3(3,4-methylenedioxyohenyl)-2-butanamine (MBDB), 3,4-methylenedioxyamphetamine (MDA), DL-3,4-methylenedioxyethylamphetamine (MDEA), methylenedioxymethamphetamine (MDMA), marijuana, mescaline, methadone, methamphetamine, methaqualone, methcathinone, morphine, noscapine, opium, oxazepam, oxycodone, phencyclidine (PCP), pentobarbital, phenobarbital, procaine, psilocybin, secobarbital, temazepam, THC, THC-COOH, and triazolam. In one embodiment, the narcotics which can be collected with a swab include cocaine, heroin, phencyclidine, THC, methamphetamine, methylenedioxyethylamphetamine, methylenedioxymethamphetamine, N-methyl-1-3(3,4-methylenedioxyohenyl)-2-butanamine, lysergic acid diethylamide, and combinations thereof.

Chemical warfare agents and other toxins that can be collected using a swab include, but are not limited to amiton (VG), anthrax, arsine, cyanogen chloride, hydrogen chloride, chlorine, diphosgene, PFIB, phosgene, phosgene oxime, chloropicrin, ethyl N,N-dimethyl phosphoramicocyanidate (Tabun), isopropyl methyl phosphonofluoridate (Sarin), pinacolyl methyl phosphonefluoridate (Soman), phosphonofluoridic acid, ethyl-, isopropyl ester (GE), phosphonothioic acid, ethyl-, S-(2-(diethylamino)ethyl) O-ethyl ester (VE), phosphonothioic acid, methyl-, S-(2-(diethylamino)ethyl) O-ethyl ester (VM), distilled mustard, ethyldichloroarsine, lewisite 1, lewisite 2, lewisite 3, methyldichloroarsine, mustard-lewisite mixture, mustard-T mixture, nitrogen mustard 1, nitrogen mustard 2, nitrogen mustard 3, phenyldichloroarsine, phosgene oxime, sesqui mustard, adamsite, aflatoxin, botulinus toxin, ricin, saxitoxin, trichothecene mycotoxin, methylphosphonothioic acid S-(2-(bis(1-methylethyl)amino)ethyl) O-ethyl ester (VX), cyclohexyl methylphosphonofluoridate (GF), and combinations thereof.

Sample analytes can be collected onto a swab by any suitable means. For example, a sample containing analytes of interest can be collected onto a swab by direct contact of the swab with the substrate to be tested or by drawing gaseous environment over or through the swab such that analytes become associated with the swab. Moreover, a swab can be manually rubbed on a substrate to be tested. Manual rubbing can be accomplished using devices and methods described in, e.g., U.S. Pat. Nos. 5,859,375 and 5,988,002. A substrate to be tested can include any person or object. For example, a substrate can be a personal effect, clothing, bag, luggage, furniture, automobile interior, etc. Alternatively, environment to be sampled can be pumped through a swab to collect a sample.

Adsorption and absorption of analytes onto a swab should be at least partially reversible. Accordingly, an analyte should be capable of being at least partially desorbed from a swab on which the analyte is adsorbed and/or absorbed. An analyte can be desorbed from a swab by any means appropriate for a given detection technique. By this, it is meant that a swab can be treated in any way necessary to prepare a sample for analysis. This treatment can depend, in part, on the type of analytes present in a sample and on the detection technique. Analytes can be desorbed from a swab though mechanical or thermal means. In one embodiment, an analyte can be desorbed from a swab by means of thermal desorption, wherein a swab is heated to vaporize the analyte. Analytes can also be desorbed from a swab by extraction of an analyte from a swab into a solvent. Without limitation, any suitable solvent can be used. Analyte-containing solvent can then be transferred to a detection instrument by any suitable means such as, for example, a syringe.

In one embodiment, analytes in a sample for analysis by ion mobility spectrometry are desorbed from a swab using thermal desorption.

B. Sampling Swab Strength and Durability

A swab suitable for use in trace analyte collection and detection should be durable and capable of resisting decomposition or degradation due to heating and mechanical stress. The resistance of a swab to decomposition and degradation when subjected to repeated mechanical and temperature stress is affected by physical properties of materials used, such as fiber strength, fiber length and fiber diameter. The strength of a swab is also dependent upon the thread count of the swab.

As used herein, “swab” and “sampling swab” are used interchangeably. “Swab” and “sampling swab” refers to a cellulosic fabric, woven or non-woven, of any size suitable for the intended application. “Cellulosic” refers to any cellulose-derived fibers and fabrics, including, but not limited to cotton, linen, rayon, flax or blends thereof. In one embodiment, the cellulosic fabric or fiber is cotton. In another embodiment, the cellulosic fabric is woven. The shape of the swab can be, without limitation, circular, oval, square, rectangular, or any other shape suitable to purpose of the swab.

One factor contributing to fabric strength is fiber length. Swabs are comprised of fibers of at least 0.8 inches in length. The fibers can be between 0.8 inches to 2.0 inches in length. In one embodiment the fibers are between 0.9 to 1.4 inches in length. In another embodiment the fibers are between 1.0 to 1.4 inches. In a further embodiment, the fibers are at least 1.2 inches in length.

Fiber strength is the measure of the strength of an individual fiber. It is commonly held that cotton fiber with a fiber strength below 23 g/tex is weak, while 24-25 g/tex is intermediate strength, 26-28 g/tex is average strength, 29 to 30 g/tex is strong, and above 31 g/tex is very strong. In one embodiment a swab is comprised of fibers having a fiber strength of at least 28 g/tex. In another embodiment, a swab is comprised of fibers having at least 31 g/tex.

Fiber strength is also expressed as a function of “bursting strength” (assessed using method ASTM D3784 or CAN/CGSB 4.2 No. 11.1) and as a function of “breaking force” (assessed using ASTM D5034 or CAN/CGSB 4.2 No. 9.2). Bursting strength is typically approximately 110 psi and breaking force is typically approximately 57.3×65.5 lb.

Another factor that contributes to fabric strength is fiber fineness, which can be indicated by micronaire value. Micronaire is a measure of specific surface area calibrated in terms of linear density. Gordon, CSIRO Textile and Fibre Technology, “An Odyssey in Fibres and Space,” Textile Institute 81^{st} World Conferences, Melbourne, Australia, April 2001. Micronaire value varies with fiber fineness and maturity. Id. Fiber fineness affects processing performance and the quality of the end product in several ways. In general, the finer of the fiber, the better the absorbency. Additionally, fabrics made from finer fiber result in more fibers per cross-section, yields stronger fabrics. Moreover, finer fibers are processed at lower speeds, which reduces damages to the fibers during processing, increasing individual fiber strength and length.

Fibers having a micronaire value of 3.7-4.2 are considered premium, while fibers having a micronaire reading of 4.34.9 and >5 are considered base range and discount, respectively. In one embodiment, the micronaire value of the fibers used in manufacturing a swab are less than approximately 5 μg/in, less than approximately 4 μg/in, less than approximately 3.5 μg/in, less than approximately 3.0 μg/in, or less than approximately 2 μg/in.

Thread count is the number of threads per square inch, and like finer fiber, higher thread counts generally result in stronger fabrics. In one embodiment a cellulosic fiber is manipulated into a fabric having a thread count of at least 40×40 threads per square inch. In another embodiment, a cellulosic fiber is manipulated into a cellulosic fabric having a thread count of at least 80×80 threads per square inch.

The presence and amount of neps is an indicator of the strength of a fiber. Moreover, neps that extend beyond the horizontal plane of a swab are more susceptible to mechanical stress and dislodgement upon mechanical stress resulting in free debris which can interfere with detection of an analyte and/or fouling of a detection instrument. As used herein, neps refer to both biological neps and mechanical neps. Biological neps are clumps of immature fibers that exist in a cellulosic fiber before processing, while mechanical neps which result from mechanical stress on a fiber during processing. In one embodiment, a swab contains fewer than 1 neps/in². In another embodiment, a swab contains fewer than 1 neps/in² extending substantially beyond the horizontal plane of a swab.

Like neps, extractables affects the mechanical strength and structural integrity of a swab. Extractables include non-lint materials trapped in the cotton, such as leaf, bark, seed-coat fragments, dust, and oil. In one embodiment, a swab can have an extractables content of less than 3%. In another embodiment, a swab can have an extractables content of less than 2%, less than 1.5%, less than 1.0%, less than 0.8%, less than 0.6%, less than 0.4%, less than 0.2%, or less than 0.1%.

C. Factors Contributing to Sampling Swab Performance

The ability of a swab to absorb and/or adsorb analytes upon contact with a substrate to be tested and efficiently desorb analytes when placed in a detection instrument is affected, in part, by the air permeability, weight per unit area and thickness of a swab.

A swab should have suitable air permeability. The air permeability of a substance is a measure of its ability of air to pass through the fabric at a predetermined rate. Suitable air permeability is useful in detection techniques where a gas is pushed through the swab to sweep analytes from the swab into the detection instrument. For example, in IMS, the swab is place into the instrument, a desorber heater vaporizes the sample, which is swept by a gas flow into an ionization region where the analytes are ionized. If a swab does not have sufficient air permeability, an IMS instrument can experience a pressure fault causing instrument failure.

Suitable swab air permeability is the range of approximately 80 cubic feet/minute (CFM) to approximately 125 CFM. In one embodiment, the air permeability is approximately 88 CFM. In another embodiment, the air permeability is approximately 115 CFM.

Air permeability can be measured by any suitable means. For example, air permeability can be measured using the standard methods provided in ASTM D737 and CAN/CGSB 4.2 No. 36.

Porosity is a function of the size and frequency of pores in a fabric. Pores, minute channels or open spaces in a solid substance, aid in adsorption or absorption of an analyte onto a swab and retention of analytes upon contact. densometer. Densometers measure the time required for a given volume of air to flow through a standard area of material being tested. Densometers are an accepted standard for measuring the porosity, air-permeability and air-resistance of sheet-like and woven materials.

The density and thickness of a swab also can affect both the collection and desorption efficiency as well as the swabs durability. A swab which is too dense or too thick can have an unacceptably high heat capacity, which can result in a poor desorption efficiency. Density is a function of weight per unit area and thickness. A suitable swab can have a weight per unit area of between approximately 0.010 g/cm² to approximately 0.02 g/cm². In one embodiment a swab has a weight per unit area of between approximately 0.012 g/cm² and approximately 0.016 g/cm². In another embodiment, a swab has a weight per unit area of approximately 0.012 g/cm². A swab can also have a thickness of between approximately 0.01 cm to approximately 0.03 cm. In one embodiment, a swab as a thickness of between approximately 0.01 cm to approximately 0.015 cm. In another embodiment, a swab has a thickness of approximately 0.012 cm. In a further embodiment, a swab has a thickness of approximately 0.025 cm. Weight per unit area, thickness and density can be determined by any means known in the art such as, for example, measurement using a densometer.

The stability of swab fiber at high temperatures is particularly important in detection methods which involve heating the swab. For example, in ion mobility spectrometry, a sampling swab is heated to desorb and vaporize sample particles collected by contact of the swab with a tested substrate. Thus, it is desirable that a swab be resistant to decomposing or degrading at high temperatures.

Although it is desirable for a swab to be stable at certain temperatures indefinitely, the stability a swab at temperatures disclosed by the present invention refers to the stability of the cotton swab at the specific temperature for at least 10 seconds, 1 minute, at least 2, minutes, at least 4 minutes, at least 6 minutes, at least 8 minutes, at least 10 minutes, at least 15 minutes, at least 20 minutes, at least 25 minutes, at least 30 minutes, or at least 1 hour. This time refers to the time over the duration of one exposure or over the duration of the usable lifetime of the swab.

In one embodiment, a swab resists degradation up to a temperature of at least 300° C., at least 325° C., or at least 350° C. In another embodiment, a swab resists degradation up to a temperature of approximately 300° C. for approximately 1 second to approximately 5 seconds. In a further embodiment, a swab resists degradation up to a temperature of approximately 300° C. for approximately 2 seconds.

D. Method of Producing Sampling Swabs

The inventors also discovered a method of processing cellulosic fibers that results a swab having properties that are particularly useful for collecting samples for trace analyte detection. The method produces a swab essentially free of impurities that can interfere with trace analyte detection, but which is capable of withstanding repeated mechanical and thermal stress without degradation or loss of structural integrity.

Cellulosic fibers can be produced using any method known in the art. Because manipulation of cellulosic fibers into fabrics exposes the fiber to considerable mechanical strain, a sizing agent can be used to protect fibers from abrasion and other rigorous mechanical actions which can impact the strength of fibers. Any appropriate sizing agent, natural or synthetic, can be used. Sizing agents include, but are not limited to starch, modified starch, vinyl, synthetic vinyl, polyvinylalcohol, polyacrylic acid, polyacrylated, carboylmethyl cellulose, polyglycol ether, and waxes. In one embodiment, the sizing agent is a starch. In another embodiment, the sizing agent is a modified starch. In a further embodiment, the sizing agent is a modified corn starch.

In another embodiment, the sizing agent is a natural or synthetic vinyl or vinyl derivative. Suitable natural and synthetic vinyls and vinyl derivatives include, but are not limited to polyvinyalcohol, polyacrylic acid, polyacrylate, carboymethyl cellulose, polyglycol ether, vinyl acetate, and polyether-based sizes, waxes and blends of these materials.

Cellulosic fibers can be manipulated into a cellulosic fabrics using any suitable method. In one embodiment the cellulosic fiber is cotton. The resulting fabric can be woven or non-woven. In one embodiment the fabric is woven. In another embodiment, the fabric is woven cotton fabric. In a further embodiment, the fabric is woven cotton fabric having a thread count of at least 80×80.

If a sizing agent is used prior to manipulating a fiber into a fabric, the sizing agent, as well as other contaminants such as inherent waxes can be removed through a process of scouring. Scouring removes sizing agents as well as fats, waxes, starches, insoluble calcium, magnesium, iron, salts of pectins, and other non-cellulosic materials from cellulosic fabric. During scouring, natural waxes and fats in the cotton fibers are saponified and pectins and other non-cellulosic materials are released.

Scouring of cellulosic fabric can be accomplished by any means known in the art, using, for example, chemical or enzymatic scouring agents. See, e.g., U.S. Pat. Nos. 4,312,634, 4,076,500, 5,912,407, 6,551,358, and 6,630,342. Suitable chemical scouring agents include, but are not limited to, caustic soda, soda ash, sodium hydroxide, potassium hydroxide, trisodium phosphate, sodium bromate, hydrochloric acid, sodium percarbonate, perborates, sodium carbonate, sodium silicate and combinations thereof. In one embodiment, from approximately 1.5% to approximately 1.0% sodium hydroxide is used as a scouring agent.

In one embodiment, a surfactant can be added in combination with a scouring agent to remove unsaponifiable materials waxes and dirt. Surfactants are typically present at a concentration of between approximately 0.01% to approximately 0.1% by weight.

Suitable surfactants include, without limitation, nonionic (see, e.g., U.S. Pat. No. 4,565,647), anionic, cationic, and zwitterionic surfactants (see, e.g U.S. Pat. No. 3,929,678). Anionic surfactants include, without limitation, linear alkylbenzenesulfonate, α-olefinsulfonate, alkyl sulfate (fatty alcohol sulfate), alcohol ethoxysulfate, secondary alkanesulfonate, α-sulfo fatty acid methyl ester, alkyl- or alkenylsuccinic acid, and soap. Nonionic surfactants include, without limitation, alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, and N-acyl N-alkyl derivatives of glucosamine (“glucamides”). Combinations of surfactants are also contemplated.

Other chemicals can also be added in combination with scouring agents, including chelating agents and other detergent builders. Chelating agents remove polyvalent metal ions such as calcium, magnesium, iron or other salts that can have a harmful effects on processing operations. Detergent builders include, for example, polymeric materials that also can act as pickup enhancing agents for use in, for example, continuous preparation.

Chelating agents and detergent builders include, without limitation, aluminosilicates, silicates, polycarboxylates and fatty acids, ethylenediamine tetraacetate, and other metal ion sequestrants such as aminopolyphosphonates, particularly ethylenediamine tetramethylene phosphonic acid and diethylene triamine pentamethylenephosphonic acid, Detergent builders and chelating agents can be included at a concentration of between approximately 5% to 80% by weight. In one embodiment, detergent builders and chelating agents are included at a concentration of between approximately 5% and approximately 30% by weight.

It can also be desirable to employ bleaching systems in the manufacture of swabs. Bleaching systems may comprise a H₂O₂ source such as perborate or percarbonate, which can be combined with a peracid-forming bleach activator such as tetraacetylethylenediamine or nonanoyloxybenzenesulfonate. Alternatively, the bleaching system may comprise peroxyacids of, e.g., the amide, imide, or sulfone type.

Other compounds that can be useful in the scouring process include antifoam agents such as, without limitation, silicones (see, e.g. U.S. Pat. No. 3,933,672) and DC-544 (Dow Corning), which are typically included at a concentration of between approximately 0.01% and approximately 1% by weight.

The scouring compositions can also contain soil-suspending agents, soil-releasing agents, optical brighteners, abrasives, and/or bactericides, as are conventionally known in the art.

Any enzyme suitable for scouring and preparing cellulosic fibers can be used. Suitable enzymes include, without limitation, pectin-digesting enzymes, proteases, lipases, cellulases, and amylases.

Suitable pectin-digesting enzymes include, without limitation, pectin-degrading enzymes such as pectin lyase (E.C. 4.2.2.2), pectin methyl esterase, polygalacturonase (E.C. 3.2.1.15), and rhamnogalacturonase (WO 92/19728); and hemicellulases such as endo-arabinanase (E.C. 3.2.1.99, Rombouts et al., Carb. Polymers 9:25, 1988), arabinofuranosidase, endo-β-1,4-galactanase, and endo-xylanase (E.C. 3.2.1.8).

Suitable proteases include those of animal, vegetable or microbial origin. In one embodiment, a protease is of microbial origin. The protease can be a serine protease or a metalloprotease. In one embodiment, the protease is an alkaline microbial protease or a trypsin-like protease. Examples of proteases include, without limitation, aminopeptidases, including prolyl aminopeptidase (E.C. 3.4.11.5), X-pro aminopeptidase (E.C. 3.4.11.9), bacterial leucyl aminopeptidase (E.C. 3.4.11.10), thermophilic aminopeptidase (E.C. 3.4.11.12), lysyl aminopeptidase (E.C. 3.4.11.15), tryptophanyl aminopeptidase (E.C. 3.4.11.17), and methionyl aminopeptidase (E.C. 3.4.11.18); serine endopeptidases, including chymotrypsin (E.C. 3.4.21.1), trypsin (E.C. 3.4.21.4), cucumisin (E.C. 3.4.21.25), brachyurin (E.C. 3.4.21.32), cerevisin (E.C. 3.4.21.48) and subtilisin (E.C. 3.4.21.62); cysteine endopeptidases, including papain (E.C. 3.4.22.2), ficain (E.C. 3.4.22.3), chymopapain (E.C. 3.4.22.6), asclepain (E.C. 3.4.22.7), actinidain (E.C. 3.4.22.14), caricain (E.C. 3.4.22.30) and ananain (E.C. 3.4.22.31); aspartic endopeptidases, including pepsin A (E.C. 3.4.23.1), aspergillopepsin I (E.C. 3.4.23.18), penicillopepsin (E.C. 3.4.23.20) and saccharopepsin (E.C. 3.4.23.25); and metalloendopeptidases, including bacillolysin (E.C. 3.4.24.28).

Suitable lipases (also termed carboxylic ester hydrolases) include those of bacterial or fungal origin, including triacylglycerol lipases (E.C. 3.1.1.3) and phospholipase A₂ (E.C. 3.1.1.4.). Commercially available lipase enzymes include Lipolase™ and Lipolase Ultra™, Lipozyme™, Palatase™, Novozym™435, and Lecitase™ (all available from Novo Nordisk A/S).

In one embodiment, amylase is used as an enzymatic scouring agent. Any suitable amylase can be used. For example, WO 94/02597, describes cleaning compositions which incorporate mutant amylases. See also WO 95/10603. Other amylases known for use in cleaning compositions include both α- and β-amylases. α-Amylases are known in the art and include those disclosed in U.S. Pat. No. 5,003,257; European Patent No. 252,666; WO 91/00353; French Patent No. 2,676,456; European Patent No. 285,123; European Patent No. 525,610; European Patent No. 368,341; and British Patent specification no. 1,296,839. Other suitable amylases are stability-enhanced amylases described in, for example, WO 94/18314 and WO 96/05295 Also suitable are amylases described in European Patent No. 277 216, WO 95/26397 and WO 96/23873. See, e.g. U.S. Pat. No. 6,677,147.

Commercially available α-amylases include Purafect Ox Am® from Genencor and Termamyl®, Ban®, Fungamyl® and Duramyl®, all available from Novo Nordisk A/S Denmark.

Suitable are variants of the above enzymes, described in WO 96/23873 (Novo Nordisk). Other amylolytic enzymes with improved properties with respect to the activity level and the combination of thermostability and a higher activity level are described in, for example, WO 95/35382.

In one embodiment, enzymes used in scouring and preparation processes are derived from alkalophilic microorganisms and/or exhibit enzymatic activity at elevated temperatures. By elevated temperatures it is meant temperatures greater than or equal to 30° C. In one embodiment, an enzyme is active at a temperature greater than or equal to 50° C. In another embodiment an enzyme is active at pH 5 to pH 9. The enzymes can be isolated from their cell of origin or may be recombinantly produced, and may be chemically or genetically modified.

An enzyme can be incorporated in a wash liquor at a concentration of from approximately 0.0001% to approximately 1% of enzyme protein by weight. In one embodiment, enzyme is present at a concentration of from approximately 0.001% to approximately 0.5%. In a further embodiment, enzyme is present at a concentration of from approximately 0.01% to approximately 0.2%.

The wash liquor and rinsing solvents can be any appropriate solvent known in the art. Suitable solvents include, but are not limited to water, chlorinated solvents, alcohols. In one embodiment, the wash liquor and rinsing solvent is water.

The step of scouring can be performed at any effective temperature or pH, which will depend, in part, on the type of scouring agent. Selection of appropriate process temperature and pH is can be determined by any suitable means and many such means are known in the art. For example, if an enzymatic scouring agent is used, at least one process in the scouring method, such as the exposure of the textile to the enzyme, should be performed at a pH and temperature where the enzyme is active. In one embodiment enzyme scouring is performed at a temperature of approximately 50 to approximately 60° C. Determination of the temperature and pH at which an enzyme is active is within the ability of a skilled artisan.

Because excessive scouring can result in broken or weakened fibers, it can be necessary to terminate scouring or select a less harsh scouring system which can result in impurities and contaminant remaining in or on the fabric. To further remove impurities, a cellulosic fabric can be heated at a temperature of between 120° C. and 250° C. for one to thirty minutes. In one embodiment, fabric is heated at a temperature of approximately 160° C. to approximately 200° C.

Heating can be performed using any means known in the art. In one embodiment, heating is performed in a forced air oven. Heating can be performed for from approximately 1 minute to approximately 60 minutes. In one embodiment, heating is performed for at least 1 minute, at least 2, minutes, at least 4 minutes, at least 6 minutes, at least 8 minutes, at least 10 minutes, at least 15 minutes, at least 20 minutes, at least 25 minutes, at least 30 minutes, or at least 60 minutes.

E. Quality Control Testing of Sampling Swabs

After processing and manufacturing of a sampling swab, either before or after the swabs are cut to size or packaged, a swab can be tested for impurities or contaminants which can interfere with the detection of the desired analyte(s) on the processed swab. Other desirable performance characteristics, such as, for example, suitable adsorption/absorption and desorption properties and general compatibility with a detection instrument, can be tested as well.

A swab can be tested for purity by analyzing a clean swab using any suitable detection method. A swab can be tested for desirable performance characteristics by placing a known analyte sample onto the swab and analyzing the known swab using a suitable detection method. Results obtained from a known analyte sample can be compared to acceptable minimum standards for certification of acceptable quality.

Swabs can be tested using any appropriate method. For example, it can be desirable to test a swab using the detection method for which the swab is intended. In one embodiment, a swab is tested using ion mobility spectrometry.

The following examples are given to illustrate the present invention. It should be understood, however, that the present invention is not to be limited to the specific embodiments described in these examples. It will be apparent to those skilled in the art that various modifications and variations can be made to the embodiments of the present invention without departing from the spirit or scope of the present invention. Thus, it is intended that the present invention covers other modifications and variations of this invention within the scope of the appended claims and their equivalents.

### EXAMPLE 1

### Quality Control Evaluation of Sampling Swabs for Explosives Detection

In this example, sampling swabs are quality control tested for use in explosives detection to ensure that (1) the swabs do not contain any contaminants which interfere with the detection of trace explosive analytes in control samples (purity) and (2) the swabs perform properly using a sample containing known analytes (performance). A clean prepared swab is analyzed in an IONSCAN® 400B ion mobility spectrometer (Smiths Detection) using the following parameters: negative ionization mode, drift tube temperature of 111° C., inlet temperature of 240° C., desorber temperature of 225° C. The ionization reagent is hexachloroethane, the drift gas is cleaned, dried room air at a flow rate of 300 cm³/min. The scan period is 22 ms with a 0.200 ms shutter gate pulse, 0.025 s analysis delay, 6.600 s analysis duration, 20 co-added scans per segment, and 15 segments per analysis.

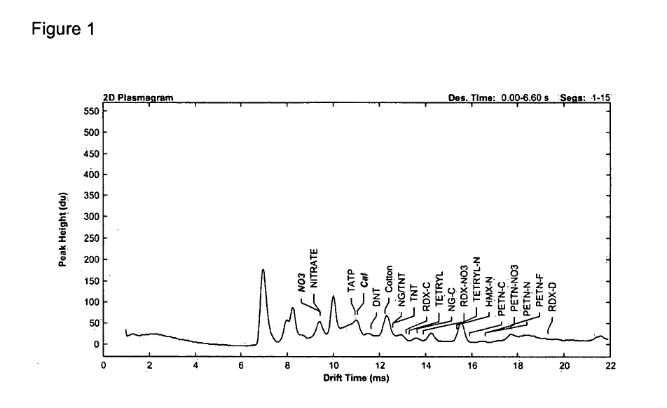

FIG. 1 is an exemplary plasmagram of a clean sample swab having acceptable quality control parameters. A swab showing acceptable purity for use in explosives trace detection should not have background peaks within the region of the drift time associated with the explosives ion peaks with an intensity greater than half of the threshold intensities for the relevant explosive ion peak. In other words, the background peaks should not exceed 50% of the values provided in following table.

| | Maximum Allowable Signal |
|---|---|
| Explosive Compound | (approx) |
| DNT | 50 |
| HMX | 25 |
| HMX-C | 25 |
| HMX-N | 25 |
| NG-C | 50 |
| NG-N | 25 |
| NG/TNT | 50 |
| NO₃ | 250 |
| PETN-C | 25 |
| PETN-F | 65 |
| PETN-N | 15 |
| RDX-C | 25 |
| RDX-D | 40 |
| RDX-F | 65 |
| RDX-N | 15 |
| Tetryl | 50 |
| Tetryl-C | 25 |
| Tetryl-N | 25 |
| TNT | 50 |

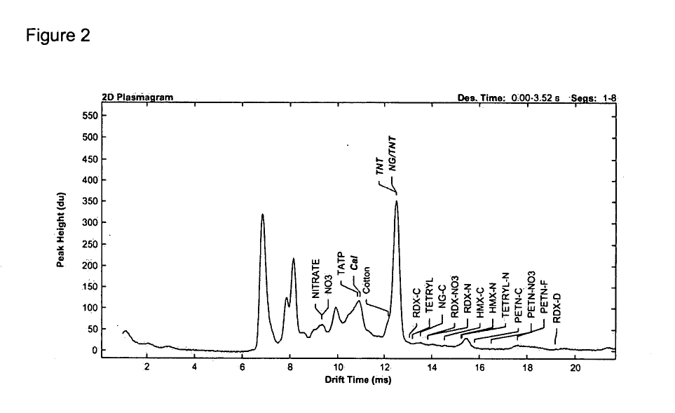

Once a lot is certified as having acceptable purity quality control parameters, the lot is also tested to ensure proper performance with control analyte samples. To determine suitability for use with explosive, a 1 μl sample containing of a 600 μg/μl TNT in hexane (600 pg TNT) is placed onto a sample using a 10 μl Hamilton syringe. The sample-containing swab is placed into the sample compartment of an IONSCAN® 400B ion mobility spectrometer and run with the following instrument parameters: negative ionization mode, drift tube temperature of 111° C., inlet temperature of 240° C., desorber temperature of 225° C. The ionization reagent is hexachloroethane, the drift gas is cleaned, dried room air at a flow rate of 350 cm³/min. The scan period is 22 ms with a 0.200 ms shutter gate pulse, 0.025 s analysis delay, 6.600 s analysis duration, 20 co-added scans per segment, and 15 segments per analysis. FIG. 2 shows an exemplary TNT plasmagram. An acceptable swab will produce a peak higher than 150 du at 12.783 ms, a characteristic drift time of TNT.

### EXAMPLE 2

### Quality Control Evaluation of Sampling Swabs for Narcotics Detection

In this example, sampling swabs are quality control tested for use in narcotics detection to ensure that (1) the swabs do not contain any contaminants which interfere with the detection of trace explosive analytes in control samples (purity) and (2) the swabs perform properly using a sample containing known analytes (performance). A clean prepared swab is analyzed in an IONSCAN® 400 B ion mobility spectrometer using the following parameters: positive ionization mode, drift tube temperature of 237° C., inlet temperature of 280° C., desorber temperature of 285° C. The ionization reagent is nicotinamide and drift gas is cleaned, dried room air at a flow rate of 300 cm³/min. The scan period is 20 ms with a 0.200 ms shutter gate pulse, 0.025 s analysis delay, 8.000 s analysis duration, 20 co-added scans per segment, and 20 segments per analysis.

A swab showing suitable purity for use in narcotics trace detection should not have background ion peaks with intensity greater than 50% of the threshold intensities for the detection of narcotics. In other words, the background at a given drift time should not exceed 50% of the values provided in the following table:

| | Maximum Allowable Signal |
|---|---|
| Narcotic Compound | (approx) |
| Cocaine | 50 |
| Hashish Mar1* | 40 |
| Hashish Mar2* | 50 |
| Hashish Mar3* | 20 |
| Hashish Mar4* | 20 |
| Heroine | 50 |
| LSD | 100 |
| MDA | 50 |
| MDEA | 100 |
| MDMA | 100 |
| Methamphetamine | 100 |
| Opium 1* | 50 |
| Opium 2* | 50 |
| Opium 3* | 50 |
| Opium 4* | 50 |
| PCP | 50 |
| Procaine | 100 |
| THC | 25 |
| VER1 | 100 |
| VER2 | 100 |
| VER3 | 100 |

*Note: Many naturally occurring narcotics demonstrate multiple peaks in the IMS plasmagram.

To determine suitability for use with narcotics, a 1 μl sample containing 1 μg/μl cocaine in hexane (1 ng cocaine) is placed onto a sample using a 10 μl Hamilton syringe. The sample-containing swab is placed into the sample compartment of an IONSCAN® 400B IMS and run with the following instrument parameters: positive ionization mode, drift tube temperature of 237° C., inlet temperature of 280° C., desorber temperature of 285° C. The ionization reagent is nicotinamide and drift gas is cleaned, dried room air at a flow rate of 300 cm³/min. The scan period is 20 ms with a 0.200 ms shutter gate pulse, 0.025 s analysis delay, 8.000 s analysis duration, 20 co-added scans per segment, and 20 segments per analysis.

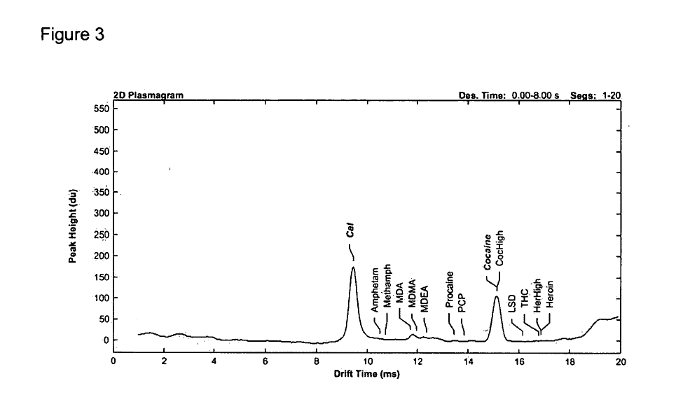

FIG. 3 shows an exemplary cocaine plasmagram. An acceptable swab will produce a peak higher than 220 du at 15 ms, a characteristic drift time of cocaine.

While the invention is described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention. All references and publications cited herein are incorporated by reference in their entireties.

1. A sampling swab comprising a cellulosic fabric comprising:
a thread count of at least 80×80;
a weight per unit area of between approximately 0.01 to approximately 0.02 g/cm²;
a thickness of between 0.01 to approximately 0.03 cm; and
an air permeability of between approximately 80 to approximately 125 CFM.

2. The sampling swab of claim 1, wherein the weight per unit area is approximately 0.012 g/cm² and wherein the thickness is from approximately 0.025 cm.

3. The sampling swab of claim 1, wherein the cellulosic fabric comprises cotton, linen, rayon, or flax or blends thereof.

4. The sampling swab of claim 3, wherein the cellulosic fabric comprises cotton.

5. The sampling swab of claim 1, wherein the cellusoic fabric has a micronaire value of less than or equal to approximately 5 μg/in.

6. The sampling swab of claim 1, wherein the cellulosic fabric comprises pores having a pore size of less than or equal to approximately 0.04 mm.

7. The sampling swab of claim 1, wherein the cellulosic fabric comprises cellulosic fibers having a bursting strength of approximately 110 psi and a breaking force of approximately 57.3×65.5.

8. The sampling swab of claim 1, wherein the strength of the theads of the fabric is at least approximately 28 grams per tex.

9. The sampling swab of claim 1, wherein the sampling swab is configured to remain stable and resist decomposition at temperatures greater than or equal to approximately 300° C.

10. The sampling swab of claim 1, wherein the fabric has an extractables content of less than approximately 3%.

11. The sampling swab of claim 1, wherein the swab is configured to have performance properties suitable to allow the swab to be utilized in a trace detection technique selected from the group consisting of ion mobility spectrometry, mass spectrometry, gas chromatography, liquid chromatography, high performance liquid chromatography, and combinations thereof.

12. The sampling swab of claim 11, wherein the trace detection technique comprises ion mobility spectrometry.

13. The sampling swab of claim 11, wherein the swab is configured to collect a sample that allows detection of compounds selected from the group consisting of explosive, narcotic, biological warfare agent, toxin, and chemical warfare agent.

14. The sampling swab of claim 13, wherein the explosive is selected from the group consisting of 2-amino-4,6-dinitrotoluene, 4-amino-2,6-dinitrotoluene, ammonal, ammonium nitrate, black powder, 2,4-dimethyl-1,3-dinitrobutane, 2,4-dinitrotoluene, ethylene glycol dinitrate, forcite 40, GOMA-2, hexanitrostilbene, 1,3,5,7-tetranitro-1,3,5,7-tetrazacyclooctane, mononitrotoluene, nitroglycerine, pentaerythritol tetranitrate, 1,3,5-trinitro-1,3,5-triazacyclohexane, semtex-A, Semtex-H, smokeless powder, trinitro-2,4,6-phenylmethylnitramine tetryl, 2,4,6-trinitrotoluene, trilita, and 1,3,5-trinitrobenzene.

15. The sampling swab of claim 13, wherein the narcotic is selected from the group consisting of 6-acetylmorphine, alprazolam, amobarbital, amphetamine, antipyrine, benzocaine, benzoylecgonine, bromazepam, butalbital, carbetapentane, cathinone, chloradiazepoxide, chlorpheniramine, cocaethylene, cocaine, codeine, diazepam, ecgonine, ecognine methyl ester, ephedrine, fentanyl, flunitrazepam, hashish, heroin, hydrocodone, hydromorphone, ketamine, lidocaine, lorazepam, lysergic acid diethylamide, lysergic acid, N-methyl-1-3(3,4-methylenedioxyohenyl)-2-butanamine, 3,4-methylenedioxyamphetamine, DL-3,4-methylenedioxyethylamphetamine, methylenedioxymethamphetamine, marijuana, mescaline, methadone, methamphetamine, methaqualone, methcathinone, morphine, noscapine, opium, oxazepam, oxycodone, phencyclidine, pentobarbital, phenobarbital, procaine, psilocybin, secobarbital, temazepam, THC, THC-COOH, and triazolam.

16. The sampling swab of claim 13, wherein the chemical warfare agent or toxin is selected from the group consisting of amiton, anthrax, arsine, cyanogen chloride, hydrogen chloride, chlorine, diphosgene, PFIB, phosgene, phosgene oxime, chloropicrin, ethyl N,N-dimethyl phosphoramicocyanidate, isopropyl methyl phosphonofluoridate, pinacolyl methyl phosphonefluoridate, phosphonofluoridic acid, ethyl-, isopropyl ester, phosphonothioic acid, ethyl-, S-(2-(diethylamino)ethyl) O-ethyl ester, phosphonothioic acid, methyl-, S-(2-(diethylamino)ethyl) O-ethyl ester, distilled mustard, ethyldichloroarsine, lewisite 1, lewisite 2, lewisite 3, methyldichloroarsine, mustard-lewisite mixture, mustard-T mixture, nitrogen mustard 1, nitrogen mustard 2, nitrogen mustard 3, phenyldichloroarsine, phosgene oxime, sesqui mustard, adamsite, aflatoxin, botulinus toxin, ricin, saxitoxin, trichothecene mycotoxin, methylphosphonothioic acid S-(2-(bis(1-methylethyl)amino)ethyl) O-ethyl ester, cyclohexyl methylphosphonofluoridate.

17. A method of processing cellulosic fabric comprising:
contacting a cellulosic fiber with a sizing agent;
manipulating the cellulosic fiber into a cellulosic fabric;
contacting the cellulosic fabric with a scouring agent;
contacting the cellulosic fabric with a solvent; and
heating the cellulosic fabric to a temperature between 120° C. to 250° C. for a time of between 1 to 60 minutes.

18. The method of claim 17, wherein the cellulosic fiber comprises cotton fiber and wherein the cellulosic fabric comprises cotton fabric.

19. The method of claim 17, wherein the step of manipulating comprises weaving and wherein a woven cellulosic fabric comprises a threat count of at least approximately 80×80.

20. The method of claim 17, wherein the sizing agent is selected from the group consisting of starch, modified starch, vinyl, synthetic vinyl, polyvinylalcohol, polyacrylic acid, polyacrylated, carboylmethyl cellulose, polyglycol ether, and waxes.

21. The method of claim 20, wherein the sizing agent is a modified starch.

22. The method of claim 20, wherein the modified starch is a modified corn starch.

23. The method of claim 17, wherein the scouring agent comprises an enzyme or a chemical composition.

24. The method of claim 23, wherein the chemical composition is an alkaline compound.

25. The method of claim 24, wherein the chemical composition is selected from the group consisting of caustic soda, soda ash, sodium hydroxide, potassium hydroxide, trisodium phosphate, sodium bromate, hydrochloric acid, sodium percarbonate, sodium perborate, sodium carbonate, sodium silicate and combinations thereof.

26. The method of claim 25, wherein the scouring agent is sodium hydroxide.

27. The method of claim 23, wherein the enzyme composition comprises an enzyme selected from the group consisting of pectin-digesting enzyme, protease, lipase, cellulase, amylase, and combinations thereof.

28. The method of claim 27, wherein the amylase is α-amylase or β-amylase, and wherein the step of contacting the cellulosic fabric with a scouring agent is performed at a temperature of approximately 50° C. to approximately 60° C.

29. The method of claim 27, wherein the enzyme is isolated from an alkalophilic microorganisms or wherein the enzyme is active at a temperature of greater than or equal to 30° C.

30. The method of claim 26, wherein the scouring agent further comprises a surfactant, a chelating agent or a builder system.

31. The method of claim 30, wherein the surfactant is selected from the group consisting of anionic, cationic, nonionic, and zwitterionic.

32. The method of claim 31, wherein the anionic surfactant is selected from the group consisting of, linear alkylbenzenesulfonate, α-olefinsulfonate, alkyl sulfate (fatty alcohol sulfate), alcohol ethoxysulfate, secondary alkanesulfonate, α-sulfo fatty acid methyl ester, alkyl- and alkenylsuccinic acid, soap, and combinations thereof.

33. The method of claim 31, wherein the nonionic surfactant is selected from the group consisting of alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, glucamides, and combinations thereof.

34. The method of claim 18 wherein the step of heating the cellulosic fabric performed at an oven temperature selected from the group consisting of at least 120° C., at least 130° C., at least 140° C., at least 150° C., at least 160° C., at least 180° C., at least 200° C., and at least 250° C.

35. The method of claim 18, wherein the step of heating the cellulosic fabric is performed at an oven temperature of approximately 160° C. to approximately 200° C.

36. The method of claim 18, wherein the step of heating the cellulosic fabric is performed for a length of time selected from the group consisting of at least 1 minute, at least 2, minutes, at least 4 minutes, at least 6 minutes, at least 8 minutes, at least 10 minutes, at least 15 minutes, at least 20 minutes, at least 25 minutes, at least 30 minutes, and at least 60 minutes.

37. The method of claim 18, wherein the step of heating the cellulosic fabric is performed for approximately 10 minutes.

38. The method of claim 18, wherein the cellulosic fabric is heated in a forced-air oven.

39. The method of claim 18 further comprising performing at least twice the steps of:
contacting the cellulosic fabric with a scouring agent, and
contacting the cellulosic fabric with a solvent.

40. The method of claim 18, wherein the cellulosic fiber is at least 1 inch long.

41. The method of claim 18, wherein the cellulosic fiber has a diameter of between approximately 0.2 mm to approximately 0.3 μm in diameter.

42. A sampling swab produced by the method of claim 18.

43. A sampling swab produced by steps comprising:
contacting a cotton fiber with a sizing agent;
manipulating the cotton fiber into a cotton fabric;
contacting the cotton fabric with a scouring agent;
contacting the cotton fabric with a solvent; and
heating the cotton fabric to a temperature between 120 to 250° C. for a time of between approximately 5 to 15 minutes.

44. The sampling swab of claim 43, further comprising:
a thread count of at least 80×80;
a weight per unit area of between approximately 0.01 to approximately 0.02 g/cm²;
a thickness of between approximately 0.01 to approximately 0.03 cm; and
an air permeability of between approximately 80 to approximately 125 CFM.
